(19) United States
(12) Patent Application Publication
(10) Pub. No.: US 2023/0115379 A1
(43) Pub. Date: Apr. 13, 2023

(54) DYNAMIC IMAGE PROCESSING DEVICE, DYNAMIC IMAGE PROCESSING SYSTEM, RECORDING MEDIUM, AND DYNAMIC IMAGE PROCESSING METHOD

(71) Applicant: KONICA MINOLTA, INC., Tokyo (JP)

(72) Inventors: Keisuke KOEDA, Tokyo (JP); Takeshi SAITO, Tokyo (JP); Nobuyuki MIYAKE, Yokohama-shi (JP); Kosuke FUKAZU, Tokyo (JP); Ryohei ITO, Tokyo (JP)

(73) Assignee: KONICA MINOLTA, INC., Tokyo (JP)

(21) Appl. No.: 17/951,333

(22) Filed: Sep. 23, 2022

(30) Foreign Application Priority Data
Oct. 1, 2021 (JP) 2021-162524

(51) Int. Cl. A61B 6/00; G06T 5/00

(52) U.S. Cl. CPC A61B 6/5282; G06T 5/00; G06T 2207/10116; G06T 2207/20182; G06T 2207/30004

(57) ABSTRACT

A dynamic image processing device including: a receiver configured to receive order information of dynamic image photography; an acquirer configured to acquire a dynamic image that is obtained by performing the dynamic image photography; and a hardware processor configured to select a scattered radiation component removal process to be used in the dynamic image, based on the order information.

### CROSS REFERENCE TO RELATED APPLICATIONS

The entire disclosure of Japanese Pat. Application No. 2021-162524 filed on Oct. 1, 2021 is incorporated herein by reference in its entirety.

### BACKGROUND

### Technological Field

The present invention relates to a dynamic image processing device, a dynamic image processing system, a recording medium, and a dynamic image processing method.

### Description of the Related Art

Techniques for obtaining a high-contrast image having least scattered radiation are conventionally known (for example, refer to JP 2016-202219A and JP 2014-207958A). This technique involves estimating scattered radiation components based on photographing conditions and photographic images of radiography and then subtracting the scattered radiation components from the photographic images.

In addition, there are techniques for improving a processing speed of removing scattered radiation by using similarity between frames specific to a moving image (dynamic image). In one example, JP 2016-063926A discloses a method of using a body thickness distribution that is determined with respect to one frame image, in other frame images in a similar manner.

In another example, JP 2010-188113A discloses a method of using a scattered radiation image of an adjacent projection direction, which has already been subjected to successive approximation calculation, as an initial estimation value (initial setting value) of a next successive calculation. This method is used in calculating a primary X-ray image and a scattered radiation distribution in each projection direction of CT-like imaging by successive approximation calculation.

In yet another example, JP 2019-130083A discloses a method of estimating a scattered radiation image by using result of a previous frame in a case in which an amount of variation between frames based on a sensor or an image signal is smaller than a threshold.

### SUMMARY

A scattered radiation removal process for removing scattered radiation components from a radiographic image requires a very long processing time. Specifically, for a still image, the processing time takes approximately 1 second, although depending on a size of one pixel and an effective pixel area of a panel. That is, it takes approximately 1 second only for one frame. On the other hand, a dynamic image composed of several hundreds of frames (e.g., 300 frames) is obtained by one photographing in dynamic image photography. Thus, for a dynamic image, a scattered radiation removal process similar to that for a still image takes a processing time of, for example, 300 seconds. In addition, in the case of a console of an instrument carriage, the processing speed is typically lower than that of a console of a general imaging room. For this reason, performing a scattered radiation removal process on a dynamic image in an instrument carriage takes time much longer than the above-described processing time, whereby problems originating from the processing time tend to become apparent in the instrument carriage.

In the method disclosed in JP 2016-063926A, a body thickness distribution is calculated with respect to one frame image of a moving image, and the calculated body thickness distribution is used in each of other frame images, in performing a scattered radiation removal process in an imaging room or in an instrument carriage. That is, JP 2016-063926A discloses a technique of shortening a period from obtaining a subject image to displaying an image excluding scattered radiation, in medical practice. Unfortunately, JP 2016-063926A is directed to performing the same scattered radiation removal process on every moving image, which method does not sufficiently consider the needs of medical professionals, and for example, can cause the following problems. That is, there is active research on diagnosis utilizing a dynamic analysis, among medical professionals, and the number of types of dynamic analysis is increasing accordingly. In such circumstances, performing the same scattered radiation removal process on every frame image of a dynamic image to be used in a dynamic analysis, can cause unnecessary waiting time for the scattered radiation removal process.

Also, JP 2016-202219A, JP 2014-207958A, JP 2010-188113A, and JP 2019-130083A do not disclose removal of scattered radiation from a dynamic image, which considers the needs of medical professionals.

The present invention has been made in view of the above-described problems, and an object thereof is to provide a dynamic image processing device, a dynamic image processing system, a dynamic image processing program, and a dynamic image processing method, each which enables selecting a scattered radiation component removal process appropriate for a dynamic image.

To achieve at least one of the abovementioned objects, according to an aspect of the present invention, a dynamic image processing device reflecting one aspect of the present invention is a dynamic image processing device including: a receiver configured to receive order information of dynamic image photography; an acquirer configured to acquire a dynamic image that is obtained by performing the dynamic image photography; and a hardware processor configured to select a scattered radiation component removal process to be used in the dynamic image, based on the order information.

To achieve at least one of the abovementioned objects, according to another aspect of the present invention, a dynamic image processing system reflecting one aspect of the present invention is a dynamic image processing system including a first dynamic image processing device and a second dynamic image processing device, wherein the first dynamic image processing device is the dynamic image processing device, and the second dynamic image processing device is configured to remove scattered radiation components from a dynamic image that is transmitted from the first dynamic image processing device, based on the dynamic image and information related to removal of scattered radiation components.

To achieve at least one of the abovementioned objects, according to another aspect of the present invention, a recording medium reflecting one aspect of the present invention is a non-transitory recording medium storing a computer-readable dynamic image processing program, the dynamic image processing program related to removal of scattered radiation components from a dynamic image that is obtained by dynamic image photography, the dynamic image processing program configured to cause a computer to execute: receiving that is receiving order information of the dynamic image photography; acquiring that is acquiring the dynamic image that is obtained by performing the dynamic image photography; and selecting that is selecting a scattered radiation component removal process to be used in the dynamic image, based on the order information.

To achieve at least one of the abovementioned objects, according to another aspect of the present invention, a dynamic image processing method reflecting one aspect of the present invention is a dynamic image processing method related to removal of scattered radiation components from a dynamic image that is obtained by dynamic image photography, the method including: receiving that is receiving order information of the dynamic image photography; acquiring that is acquiring the dynamic image that is obtained by performing the dynamic image photography; and selecting that is selecting a scattered radiation component removal process to be used in the dynamic image, based on the order information.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are no intended as a definition of the limits of the present invention, wherein:

FIG. 1 illustrates an example of the whole configuration of a dynamic image processing system;

FIG. 2 is a block diagram illustrating a functional configuration of a mobile radiographic apparatus in FIG. 1;

FIG. 3 illustrates an example of stored data in a photographing condition table;

FIG. 4 illustrates an example of stored data in an employed process selection table;

FIG. 5 illustrates an example of an examination screen before a scattered radiation component removal process is selected prior to photography;

FIG. 6 is a flowchart illustrating a flow of a scattered radiation removal control processing “A” that is executed by a controller in FIG. 2;

FIG. 7 illustrates an example of the examination screen after the scattered radiation component removal process is selected prior to photography;

FIG. 8 illustrates an example of the examination screen after photography is performed;

FIG. 9 illustrates an example of the examination screen after photography is performed;

FIG. 10 illustrates an example of the examination screen after photography is performed; and

FIG. 11 is a flowchart illustrating a flow of a scattered radiation removal control processing “B” that is executed by the controller in FIG. 2.

### DETAILED DESCRIPTION OF THE EMBODIMENTS

### Configuration of Dynamic Image Processing System 100

First, a configuration of an embodiment of the present invention will be described. However, the scope of the invention is not limited to the disclosed embodiments.

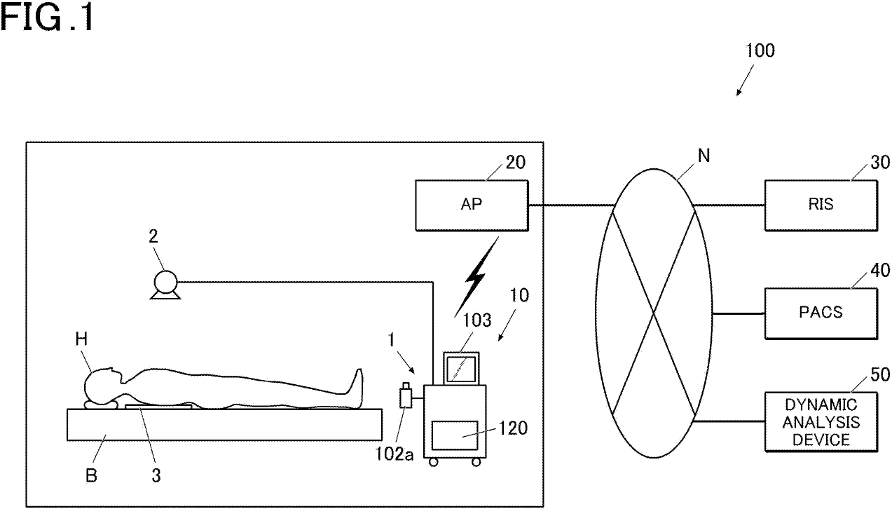

FIG. 1 illustrates an example of the whole configuration of a dynamic image processing system 100 in this embodiment. As illustrated in FIG. 1, the dynamic image processing system 100 is constructed by connecting a mobile radiographic apparatus 10, a radiology information system (RIS) 30, a picture archiving and communication system (PACS) 40, and a dynamic analysis device 50 to each other via a communication network “N”, such as a local area network (LAN) or a wide area network (WAN), in a data transmittable/receivable manner. The mobile radiographic apparatus 10 is connected to the communication network “N” via a wireless access point (AP) 20 of a wireless LAN or a wired LAN cable (not illustrated). A plurality of wireless access points 20 are provided in a medical facility in which the dynamic image processing system 100 is installed.

The mobile radiographic apparatus 10 is, for example, an apparatus for performing radiography on a patient who has a difficulty in moving, by visiting the patient. The mobile radiographic apparatus 10 includes a main body 1, a radiation source 2, and a flat panel detector (FPD) cassette 3.

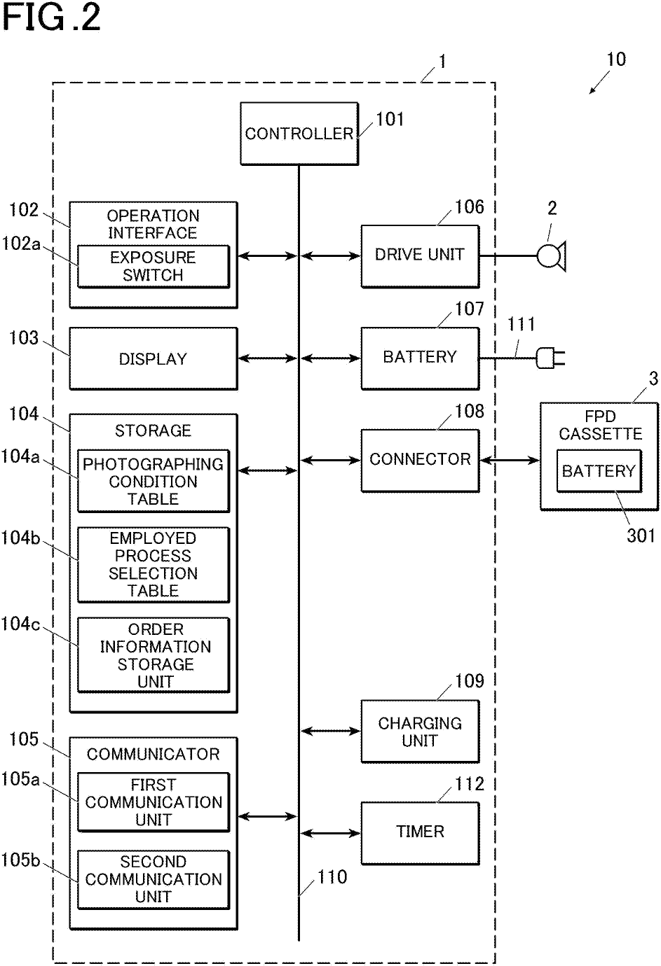

The mobile radiographic apparatus 10 has wheels on the main body 1 and is constructed as an instrument carriage that can be moved. The main body 1 is provided with a storage space 120 for storing the FPD cassette 3. The storage space 120 is provided with a connector 108 (refer to FIG. 2) for connecting the stored FPD cassette 3, and thus, the stored FPD cassette 3 can be carried while a battery 301 (refer to FIG. 2) of the FPD cassette 3 is being charged.

The mobile radiographic apparatus 10 may be a portable type that does not have wheels.

As illustrated in FIG. 1, the mobile radiographic apparatus 10 is brought into an operating room, an intensive care unit, a hospital room, or the like, and the FPD cassette 3 is set up. For example, the FPD cassette 3 is inserted between a bed “B” and a subject “H” lying on the bed “B” or into a slot (not illustrated) that is provided on a side opposite to the subject “H” on the bed “B”. In such a state, the mobile radiographic apparatus 10 performs still image photography or dynamic image photography on the subject “H” by emitting radiation from the radiation source 2. In this embodiment, the still image photography is to obtain one image of a subject in response to one photographing operation (pressing down an exposure switch 102a).

The dynamic image photography is to obtain a plurality of images of a subject in response to one photographing operation. In more detail, pulses of radiation, such as X-rays, are repeatedly emitted to a subject at a predetermined time interval (pulse irradiation), or radiation of a low dose rate is continuously emitted without interruption (continuous irradiation). The series of images that are obtained by dynamic image photography is called a “dynamic image”. In addition, each of the plurality of images constituting the dynamic image is called a “frame image”.

Herein, the dynamic image photography includes dynamic image photography, but it does not include photographing of still images while showing a moving image. In addition, the dynamic image includes a moving image, but it does not include images that are obtained by taking still images while showing a moving image.

FIG. 2 is a block diagram illustrating a functional configuration of the mobile radiographic apparatus 10.

The main body 1 of the mobile radiographic apparatus 10 has a function as a console and a dynamic image processing device (first dynamic image processing device). As illustrated in FIG. 2, the main body 1 includes a controller 101 (hardware processor), an operation interface 102, a display 103, a storage 104, a communicator 105, a drive unit 106, a battery 107, a connector 108, a charging unit 109, and a timer 112 that are connected to each other via a bus 110.

The controller 101 is composed of a central processing unit (CPU), a random access memory (RAM), and so on. The CPU of the controller 101 reads a system program and each type of processing programs, which are stored in the storage 104, in response to input from the operation interface 102, and the CPU loads them into the RAM to execute various processes in accordance with the loaded programs.

The controller 101 functions as a selection unit and a scattered radiation removal process unit.

The operation interface 102 includes a touch panel in which transparent electrodes are arranged in a grid pattern so as to cover the surface of the display 103. The operation interface 102 detects a position that is pressed down by a finger, a stylus, or the like, and it then inputs information of this position to the controller 101, as operation information.

The operation interface 102 also includes an exposure switch 102a that is used by a user to instruct start of emitting radiation.

The display 103 is composed of a monitor, such as a liquid crystal display (LCD) or a cathode ray tube (CRT), and it performs displaying in accordance with an instruction of a display signal input from the controller 101.

The storage 104 is composed of a non-volatile semiconductor memory, a hard disk drive, and so on. The storage 104 stores various programs that are executed by the controller 101, parameters necessary for programs to execute processes, and data such as result of processing.

In this embodiment, the storage 104 also stores a photographing condition table 104a and an employed process selection table 104b.

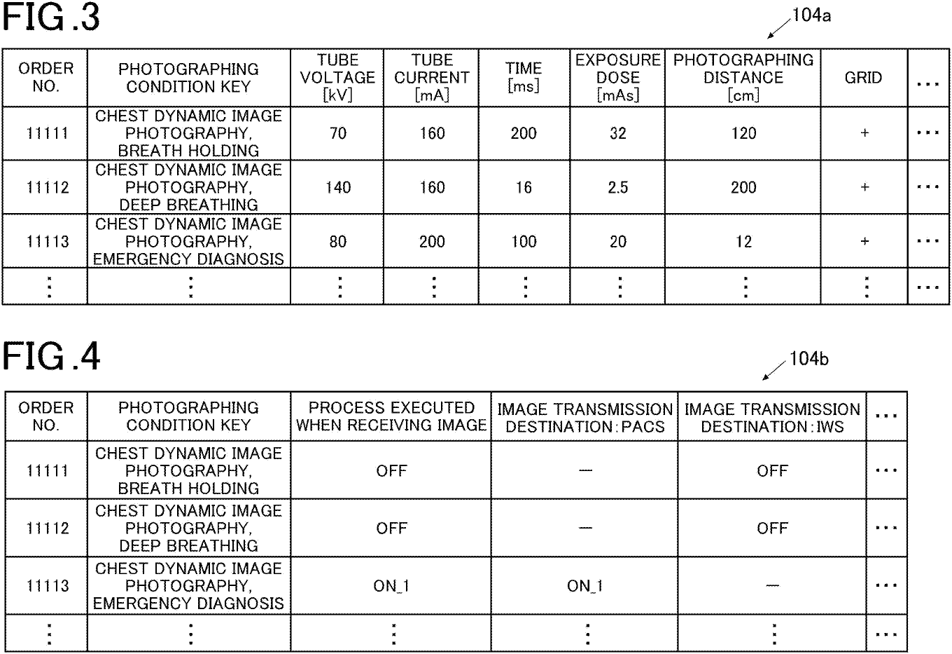

FIG. 3 illustrates an example of the photographing condition table 104a. As illustrated in FIG. 3, the photographing condition table 104a stores an order No., and a photographing condition key and photographing conditions corresponding to an order No., in association with each other. The photographing conditions include tube voltage, tube current, irradiation time, an exposure dose, a photographing distance (SID), grid information (presence/absence of grid), a frame rate, and a type of radiation detector. In this embodiment, order information that is transmitted from the RIS 30 contains an order No. for identifying contents of an order for each photography included in examination. The photographing condition key shows contents of an order corresponding to the order No. The controller 101 is able to determine contents of an order and corresponding photographing conditions, from the received order No., by referring to the photographing condition table 104a.

The order that is identified based on the order No. contained in order information about dynamic image photography, which is transmitted from the RIS 30, includes at least one of information related to the type of dynamic analysis and information related to the type of diagnosis.

The information related to the type of dynamic analysis is information that enables determining the type of dynamic analysis to be conducted on a dynamic image obtained by photographing. Examples of the information related to the type of dynamic analysis include “chest dynamic image photography, breath holding”, “chest dynamic image photography, deep breathing”, “chest dynamic image photography, deep breathing, adhesion”, and “chest dynamic image photography, deep breathing, tumor”. The “chest dynamic image photography, breath holding” represents a blood flow analysis, the “chest dynamic image photography, deep breathing” represents a ventilation analysis, the “chest dynamic image photography, deep breathing, adhesion” represents an adhesion analysis, and the “chest dynamic image photography, deep breathing, tumor” represents a tumor analysis. The type of each dynamic analysis is a macroscopic analysis or a microscopic analysis, and whether ordered analysis is the macroscopic analysis or the microscopic analysis can be determined based on the information related to the type of dynamic analysis. That is, the information related to the type of dynamic analysis contains information related to the macroscopic analysis or information related to the microscopic analysis. The macroscopic analysis is an analysis used in a diagnosis performed by looking over the whole subject, and it includes, for example, a ventilation analysis and a blood flow analysis, in the case of chest. The microscopic analysis is an analysis used in a diagnosis performed by looking at a small region of a subject, and it includes, for example, a tumor analysis and an adhesion analysis, in the case of chest.

Examples of the information related to the type of diagnosis include “emergency medical care”, “visit to patient”, “imaging room”, and “home”.

The orders that can be specified by using the RIS 30 and the type of dynamic analysis corresponding to each order are stored in the storage 104 in association with each other. For example, the “chest dynamic image photography, breath holding” is stored in association with a blood flow analysis, the “chest dynamic image photography, deep breathing” is stored in association with a ventilation analysis, the “chest dynamic image photography, deep breathing, adhesion” is stored in association with an adhesion analysis, and the “chest dynamic image photography, deep breathing, tumor” is stored in association with a tumor analysis. In addition, “chest dynamic image photography, emergency diagnosis” is stored in association with an adhesion analysis. This is because there is a need to check existence of adhesion prior to operation, in emergency medical care. Moreover, information whether the type of each dynamic analysis corresponds to a macroscopic analysis or a microscopic analysis is also stored in the storage 104.

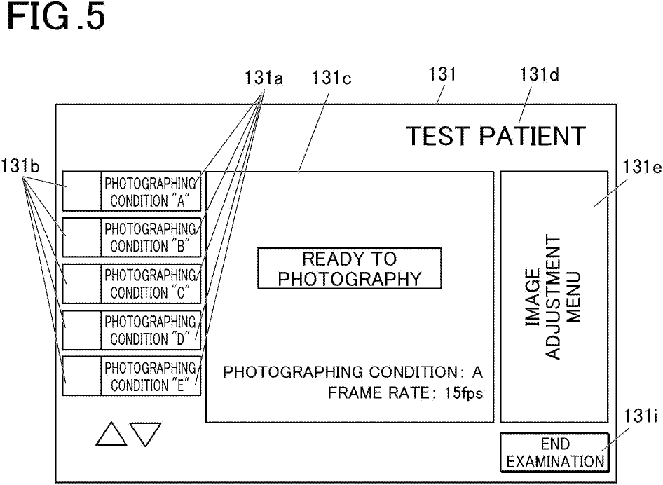

FIG. 4 illustrates an example of the employed process selection table 104b. As illustrated in FIG. 4, the employed process selection table 104b stores items “PROCESS EXECUTED WHEN RECEIVING IMAGE”, “IMAGE TRANSMISSION DESTINATION: PACS”, and “IMAGE TRANSMISSION DESTINATION: IWS” that are associated with each of an order No. and a photographing condition key corresponding to the order No.

For the item “PROCESS EXECUTED WHEN RECEIVING IMAGE”, information showing a scattered radiation component removal process that is employed at the time of receiving a dynamic image, which is obtained by photographing, is stored. For the item “IMAGE TRANSMISSION DESTINATION: PACS”, information showing a scattered radiation component removal process that is used in a dynamic image to be transmitted to the PACS 40, is stored. For the item “IMAGE TRANSMISSION DESTINATION: IWS”, information showing a scattered radiation component removal process that is used in a dynamic image to be transmitted to the dynamic analysis device 50, is stored. In each item, a term “ON_1” is stored for the case of performing a normal process of scattered radiation removal, a term “ON_2_1” is stored for the case of performing a simplified process (simple body thickness estimation), a term “ON_2_2” is stored for the case of performing a simplified process (simple scattered radiation component estimation), and a term “OFF” is stored for the case of not performing the scattered radiation removal process.

In the items “IMAGE TRANSMISSION DESTINATION: PACS” and “IMAGE TRANSMISSION DESTINATION: IWS”, a term “NULL” (which is represented by “-” in FIG. 4) is stored for the case in which the image is not transmitted to the corresponding transmission destination.

Herein, in the present application, the scattered radiation component removal process represents a process related to removal of scattered radiation components. The scattered radiation component removal process that is applicable to a dynamic image includes at least one of a normal process (first process), simplified processes (second processes) that are simplified more than the normal process, and a process that does not involve the scattered radiation removal process (third process). Details of the normal process, the simplified processes, and the process that does not involve the scattered radiation removal process will be described later.

In addition, the scattered radiation removal process represents a process of removing scattered radiation components from an image (dynamic image).

It is possible for a user to set the employed process selection table 104b by using the operation interface 102 or via a second communication unit 105b or the like, as desired.

In addition, the storage 104 is provided with an order information storage unit 104c that stores order information obtained from the RIS 30. Herein, the order information that is obtained from the RIS 30 contains, for example, an examination ID, an examination date, patient information related to a patient who will be a subject, and an order No. showing an order related to each photographing to be performed in examination. The patient information includes a patient ID, a name, gender, and age. As described above, in the photographing condition table 104a and the employed process selection table 104b, the order No. and the photographing condition key, which shows contents of an order, are associated with each other. Thus, contents of an order corresponding to an order No. of each photographing can be determined on the mobile radiographic apparatus 10 side.

The storage 104 is also provided with a temporary storage area that temporarily stores information waiting for transmission to an external device (e.g., a dynamic image (original image) or an image that has been subjected to the scattered radiation removal process).

The communicator 105 includes a first communication unit 105a and a second communication unit 105b. The first communication unit 105a transmits and receives data to and from the FPD cassette 3 by wired communication or wireless communication. The second communication unit 105b transmits and receives (inputs and outputs) data to and from an external device such as the RIS 30, the PACS 40, or the dynamic analysis device 50, which are connected to the communication network “N” via the wireless access point 20 or a wired LAN cable (not illustrated).

The second communication unit 105b functions as an acquirer, a receiver, and a transmitter.

The drive unit 106 is a circuit that drives a tubular lamp of the radiation source 2. The drive unit 106 and the radiation source 2 are connected to each other via a cable.

The battery 107 supplies power to each component of the main body 1 and the radiation source 2. The battery 107 can be charged from the outside via an AC cable 111. The battery 107 is charged in advance via the AC cable 111 during a time when photographing operation is not performed. The AC cable 111 is contained inside the main body 1 at the time of carrying.

The connector 108 is provided inside the storage space 120 and is electrically connected to the FPD cassette 3 stored in the storage space 120.

The charging unit 109 is a circuit for charging the battery 301 of the FPD cassette 3 that is connected via the connector 108, with electric power supplied from the battery 107, based on control from the controller 101 during a time of not performing photographing.

The timer 112 measures a preliminarily set time in accordance with an instruction from the controller 101 and notifies the controller 101 after the preliminarily set time has elapsed.

The radiation source 2 is driven by the drive unit 106 and emits radiation (X-rays) to a subject “H”.

The FPD cassette 3 is a portable radiation detector that has the rechargable battery 301 as a drive source, and it can be used in still image photography and in dynamic image photography. The FPD cassette 3 includes, for example, a glass substrate on which a plurality of detecting elements are two-dimensionally arranged at predetermined positions. The detecting elements detect radiation that has been emitted from the radiation source 2 and has passed through at least a subject “H”, in accordance with the intensity of the radiation. The detecting elements then convert the detected radiation into electrical signals and accumulate them. The detecting elements are composed of semiconductor image sensors, such as photodiodes. Each of the detecting elements is connected to a switching device, such as a thin film transistor (TFT), and the switching device controls accumulation and reading of electrical signals, whereby image data (frame image) is obtained.

There are an indirect conversion FPD and a direct conversion FPD. The indirect conversion FPD converts radiation into an electrical signal via a scintillator by using a photoelectric conversion element. The direct conversion FPD directly converts radiation into an electrical signal. Either the indirect conversion FPD or the direct conversion FPD can be used as the FPD cassette 3.

The RIS 30 issues and stores order information of examination and transmits the issued order information to a modality such as the mobile radiographic apparatus 10, via the communication network “N”.

The PACS 40 is an image management device that stores and manages medical images (still images and dynamic images) that are generated by a modality such as the mobile radiographic apparatus 10, and results of analysis performed by the dynamic analysis device 50, in association with patient information and examination information. The examination information includes an examination ID, examination date and time, a photographed region, and photographing conditions.

The dynamic analysis device 50 is a second dynamic image processing device. The dynamic analysis device 50 performs an analysis process, such as a dynamic analysis of a subject, on a dynamic image transmitted from the mobile radiographic apparatus 10 or other apparatus, and it then transmits the dynamic image and result of analysis to the PACS 40. The dynamic analysis device 50 supports a plurality of types of dynamic analysis and conducts a dynamic analysis of a type that is specified by order information, among the plurality of types of dynamic analysis.

### Operation of Dynamic Image Processing System 100

Next, operation of the dynamic image processing system 100 will be described.

In the state in which contents of order information are input to (specified in) the RIS 30 by a doctor or the like, in response to an instruction to issue the order information, the RIS 30 issues and transmits the order information to the mobile radiographic apparatus 10.

In the mobile radiographic apparatus 10, after the second communication unit 105b receives the order information from the RIS 30, the controller 101 makes the order information storage unit 104c store the received order information and also makes the display 103 show the order information on an examination list screen (not illustrated). The order information contains an examination ID, an examination date, patient information, and information related to each photographing included in examination (herein, an order No.). In response to selection of the order information of an examination to be conducted, in the examination list screen, the controller 101 makes the display 103 show an examination screen 131.

FIG. 5 illustrates an example of the examination screen 131. As illustrated in FIG. 5, the examination screen 131 is provided with photographing condition buttons 131a, thumbnail display areas 131b, an image display area 131c, a patient information display area 131d, an image adjustment menu area 131e, an examination end button 131i, etc.

The photographing condition button 131a is a button provided with respect to an order for each photography contained in the order information. This button is used to set photographing conditions (irradiation conditions and image reading conditions) in accordance with an order for each photography, in the radiation source 2 and in the FPD cassette 3. Each of the photographing condition buttons 131a shows a photographing condition key that represents contents of an order for each photography contained in the order information.

The thumbnail display area 131b is an area for showing a thumbnail image of a radiographic image that is obtained by radiography performed in response to pressing down the adjacent photographing condition button 131a.

The image display area 131c is an area for showing a radiographic image that is obtained by radiography.

The patient information display area 131d is an area for showing patient information of a patient (subject) to be examined.

The image adjustment menu area 131e is an area for showing an image adjustment menu for a radiographic image that is shown in the image display area 131c.

The examination end button 131i is a button for a user to instruct finishing examination.

A user can press down the photographing condition button 131a for radiography to be performed next, in the examination screen 131, to prepare for photographing.

A user may press down one of the photographing condition buttons 131a on the examination screen 131 by operating the operation interface 102. In response to this, the controller 101 reads the photographing conditions corresponding to the photographing condition button 131a that has been pressed down, from the photographing condition table 104a in the storage 104. The controller 101 then sets the irradiation conditions (e.g., tube voltage, tube current, irradiation time, an exposure dose, a photographing distance, grid information, and a frame rate) to the drive unit 106, among the read photographing conditions. In addition, the controller 101 transmits the image reading conditions (e.g., a frame rate and a pixel size) to the FPD cassette 3 from the first communication unit 105a, among the read photographing conditions.

The photographing conditions may be manually set by a user, and in this case, settings of the photographing conditions of the same patient and the same region in another photographing may be automatically used as they are. In one example in which an AP front chest still image and an AP front chest dynamic image are successively obtained by photographing, in this order, the photographing conditions for still image photography may be used as the photographing conditions for dynamic image photography, as they are. This can reduce an operation burden of a user.

Moreover, in response to a user pressing down one of the photographing condition buttons 131a on the examination screen 131 by operating the operation interface 102, the controller 101 determines whether the next photographing is dynamic image photography, based on the order information corresponding to the photographing condition button 131a that has been pressed down. Upon determining that the next photographing is dynamic image photography, the controller 101 further determines whether the photographing is performed by using a grid, based on the photographing conditions corresponding to the photographing condition button 131a that has been pressed down. In the case of determining that the photographing is not performed by using a grid (that is, the photographing is performed without a grid), the controller 101 executes a scattered radiation removal control processing “A” and a scattered radiation removal control processing “B”, in cooperation with a program stored in the storage 104.

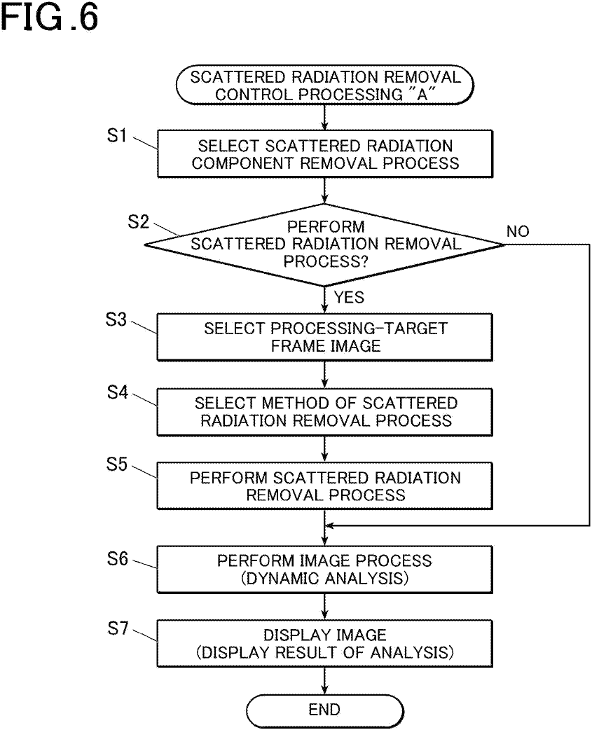

FIG. 6 is a flowchart illustrating a flow of the scattered radiation removal control processing “A”. The scattered radiation removal control processing “A” is executed by cooperation of the controller 101 and the program stored in the storage 104.

In the scattered radiation removal control processing “A”, first, the controller 101 selects a scattered radiation component removal process to be used in a dynamic image at the time of receiving the dynamic image obtained by photographing (step S1).

At this stage, typically, a still image that is obtained by photographing without a grid is subjected to a scattered radiation removal process. The scattered radiation removal process takes approximately 1 second, per still image, although depending on a size of one pixel and an effective pixel area of a panel. On the other hand, a dynamic image composed of several hundreds of frames (e.g., 300 frames) is obtained by one photographing in dynamic image photography. Thus, if a scattered radiation removal process similar to that for a still image is performed on every frame image of a dynamic image that is obtained by photographing without a grid, a long processing time, for example, 300 seconds, is required. In addition, in the case of a mobile radiographic apparatus, the processing speed is typically lower than that of a console of a general imaging room. For this reason, a processing time much longer than the above-described processing time is required, whereby problems originating from the processing time tend to become apparent.

In view of this, the inventors of the present application have investigated the need to preliminarily perform a scattered radiation removal process similar to that for a still image, on a dynamic image that is obtained by photographing without a grid, prior to a dynamic analysis.

As a result, the investigation that is conducted by the inventors of the present application reveals the following findings: Preliminarily performing a scattered radiation removal process on a dynamic image does not greatly improve analysis accuracy and is unnecessary in many cases, in a macroscopic analysis such as a ventilation analysis or a blood flow analysis, which is used for looking over the whole subject (herein, a lung field) to make a diagnosis, among dynamic analysis using a dynamic image. In addition, it is revealed that scattered radiation components can be removed by obtaining a difference between signal values, in analysis such as a ventilation analysis or a blood flow analysis, which calculates a difference of a signal value between frame images.

On the other hand, the investigation that is conducted by the inventors of the present application also reveals that a scattered radiation removal process has a great effect for improving analysis accuracy and visibility of a target (e.g., a tumor or adhesion) in visual observation in the microscopic analysis. The microscopic analysis is used for looking at a small region of a subject to make a diagnosis. The microscopic analysis involves a rib suppression process and a frequency enhancement process and can determine a microscopic region (e.g., a tumor or adhesion) that moves in a lung field in synchronization with breathing movement of a patient. Moreover, the investigation that is conducted by the inventors of the present application reveals that, in order to recognize adhesion, in which existence is desired to be checked prior to operation (for example, in emergency medical care), it is necessary to perform an adhesion analysis (microscopic analysis) for analyzing movement of a microscopic region in a lung field.

Both of the dynamic image for the macroscopic analysis and the dynamic image for the microscopic analysis are obtained by photographing in the condition in which positioning of a patient is set to positioning called “front chest”. The need for performing the scattered radiation removal process depends on the type of the dynamic analysis, as described above. Thus, conducting the scattered radiation removal process on a dynamic image to be subjected to a dynamic analysis that does not require the scattered radiation removal process, causes an unnecessary processing time. This reduces working efficiency of a radiographer (user) and a diagnosis doctor and increases waiting time of a patient who is ready for photography. At the same time, a process of subtracting scattered radiation components, which is specific to the scattered radiation removal process, increases noise, resulting in undesirable deterioration in image quality.

For a dynamic image that is used in the case of visiting a patient, some medical professionals want to perform a diagnosis based on result of analyzing a dynamic image that has been subjected to the scattered radiation removal process (for example, want to diagnose result of analyzing adhesion), immediately after photography. On the other hand, some medical professionals want to perform only photographing during visit to a patient and to make a diagnosis after visit to the patient. From this point of view, if a scattered radiation removal process similar to that for a still image is performed on every frame image of a dynamic image, during visit to a patient, the latter users have to wait for completion of the scattered radiation removal process occurs.

In consideration of this, in step S1, the controller 101 automatically selects the scattered radiation component removal process to be used in a dynamic image by the mobile radiographic apparatus 10, based on the order information corresponding to the photographing condition button 131a that has been pressed down in the examination screen 131. The process is selected from among the normal process, the simplified processes that are simplified more than the normal process, and the process that does not involve the scattered radiation removal process.

The normal process is a process for performing all basic processes on every frame image to be subjected to the scattered radiation removal process of a dynamic image. The basic processes include body thickness estimation based on a corresponding frame image, scattered radiation component estimation, and subtraction of scattered radiation components from the frame image. The advantage of this process is having high accuracy of estimation of scattered radiation components due to estimation of body thickness and scattered radiation components in every frame image. The disadvantage is that the processing time is long due to performing all steps of the scattered radiation removal process on every frame image.

The simplified processes are processes of executing all of the basic processes on one or some of frame images to be subjected to the scattered radiation removal process of a dynamic image, and executing a simple scattered radiation removal process on the other frame images. The simple scattered radiation removal process uses a parameter (body thickness or scattered radiation component) for removing scattered radiation components, which is estimated (obtained) from the frame image that has been subjected to the basic processes. A process of estimating a body thickness from one or some of frame images and then performing estimation and subtraction of scattered radiation components on the other frame images by using the body thickness that is estimated from the one or some of the frame images, is called a “simplified process (simple body thickness estimation)”. A process of estimating scattered radiation components from one or some of frame images and then performing subtraction of scattered radiation components on the other frame images by using the scattered radiation components that are estimated from the one or some of the frame images, is called a “simplified process (simple scattered radiation component estimation)”. The advantage of the simplified processes is that the processing time is shorter than that of the normal process. The disadvantage is that the estimation accuracy is lower than that of the normal process.

The process that does not involve the scattered radiation removal process is a process of not performing the scattered radiation removal process on a dynamic image in the apparatus that performs this process (mobile radiographic apparatus 10). The scattered radiation removal process may be performed in an external device that is a transmission destination of a dynamic image (e.g., the dynamic analysis device 50).

In one example, the controller 101 refers to the item “PROCESS EXECUTED WHEN RECEIVING IMAGE” of an order corresponding to the photographing condition button 131a that has been pressed down, in the employed process selection table 104b. In the case in which the information in this item is the term “ON_1”, the normal process is selected. In the case in which the information in this item is the term “ON_2_1”, the simplified process (simple body thickness estimation) is selected.

In the case in which the information in this item is the term “ON_2_2”, the simplified process (simple scattered radiation component estimation) is selected.

In the case in which the information in this item is the term “OFF”, the process that does not involve the scattered radiation removal process is selected.

The employed process selection table 104b is generated based on the investigation described above. For example, as illustrated in FIG. 4, the term “OFF” is stored in the item “PROCESS EXECUTED WHEN RECEIVING IMAGE” corresponding to the order containing information related to the macroscopic analysis. In the item “PROCESS EXECUTED WHEN RECEIVING IMAGE” corresponding to the order containing information related to the microscopic analysis, the term “ON” (“ON_1”, “ON_2_1”, or “ON_2_2”; the same applies to the following description) is stored.

In the item “PROCESS EXECUTED WHEN RECEIVING IMAGE” corresponding to the order containing information related to emergency medical care, the term “ON” is stored. In the item “PROCESS EXECUTED WHEN RECEIVING IMAGE” corresponding to the order that does not contain information related to emergency medical care, the term “OFF” is stored.

In short, the controller 101 selects the process that does not involve the scattered radiation removal process, for the order information containing information related to the macroscopic analysis. For the order information containing information related to the microscopic analysis, the normal process or the simplified process is selected. It is more preferable for the mobile radiographic apparatus 10 that is used as a radiographic apparatus, to select the simplified process. This is because the controller of the mobile radiographic apparatus 10 has a performance lower than that of a photographing apparatus used in a radiology room, and the processing time is presumed to be long. On the other hand, a stationary radiographic apparatus that is used as a radiographic apparatus may select the normal process due to including a controller having a performance higher than that of a mobile radiographic apparatus. For the order information containing information related to emergency medical care, the normal process or the simplified process is selected. It is more preferable for the mobile radiographic apparatus 10 that is used as a radiographic apparatus, to select the simplified process. The reason of this is as follows. In a case of performing dynamic image photography on a patient carried by an ambulance, on the spot, such as in an operating room, although the controller of the mobile radiographic apparatus 10 has a performance lower than that of a photographing apparatus used in a radiology room, as described above, it is necessary to rapidly obtain information required in operation. On the other hand, a stationary radiographic apparatus that is used as a radiographic apparatus may select the normal process due to including a controller having a performance higher than that of a mobile radiographic apparatus. For the order information that does not contain information related to emergency medical care, the process that does not involve the scattered radiation removal process is selected.

Note that the employed process selection table 104b illustrated in FIG. 4 is an example only and can be set by a user, as desired. In one example, an order may contain information related to the microscopic analysis, such as “chest dynamic image photography, deep breathing, adhesion” or “chest dynamic image photography, deep breathing, tumor”. However, in the case in which a dynamic image is transmitted to the dynamic analysis device 50 in a situation other than emergency medical care, the term “OFF” may be stored in the item “PROCESS EXECUTED WHEN RECEIVING IMAGE”. On the other hand, it is necessary to check a dynamic image or analysis result on the spot, also in the cases of “visit to patient” and “home”, in addition to the case “emergency medical care”. Thus, also for an order containing information related to diagnosis, such as “visit to patient” or “home”, the term “ON” may be stored in the item “PROCESS EXECUTED WHEN RECEIVING IMAGE”.

After selecting the scattered radiation component removal process to be used in a dynamic image, the controller 101 stores the result of selection in the RAM and shows the result of selection on the examination screen 131, prior to photography.

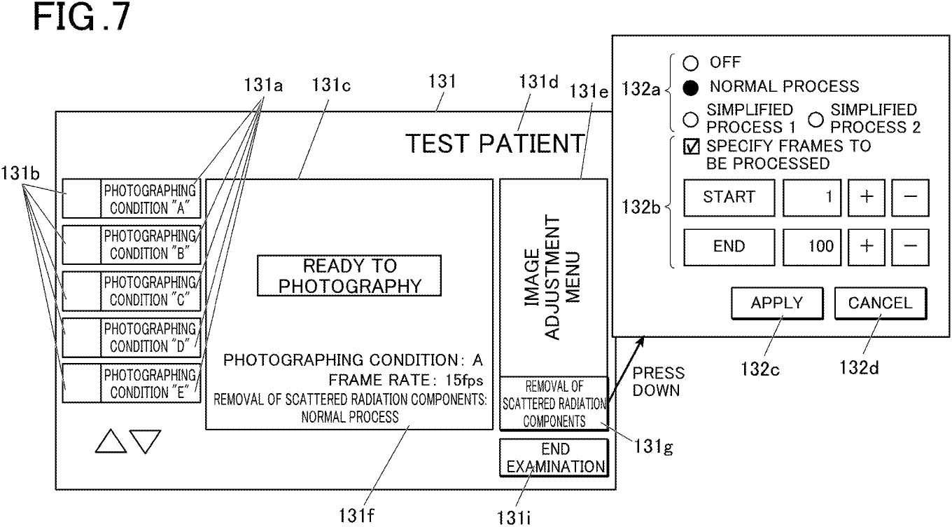

FIG. 7 illustrates an example of the examination screen 131 that shows the result of selecting the scattered radiation component removal process. An image display area 131c of the examination screen 131 illustrated in FIG. 7 shows process information 131f that shows the selected scattered radiation component removal process. In addition, the image adjustment menu area 131e shows a scattered radiation component removal menu 131g.

In response to the scattered radiation component removal menu 131g being pressed down by using the operation interface 102, the controller 101 pops up a change screen 132 for a user to change the scattered radiation component removal process to be used in a dynamic image.

The change screen 132 is provided with radio buttons 132a, a column 132b for specifying processing-target frames, an apply button 132c, and a cancel button 132d. The radio buttons 132a are used for selecting the radiation component removal process from among “OFF”, “NORMAL PROCESS”, “SIMPLIFIED PROCESS 1 (SIMPLE BODY THICKNESS ESTIMATION)”, and “SIMPLIFIED PROCESS 2 (SIMPLE SCATTERED RADIATION COMPONENT ESTIMATION)”. The column 132b for specifying processing-target frames is used for specifying a start frame number and a finish frame number of processing-target frames. Unless the checkbox is checked, all frame images are selected as processing targets.

After the settings are changed, and the apply button 132c is pressed down, the controller 101 changes the result of selection stored in the RAM. That is, the scattered radiation component removal process to be used in a dynamic image is changed.

The radiation source 2 may be provided with an optical camera, and presence/absence of a grid in photographing may be detected in a photographic image obtained by the optical camera. In the case in which detected information of presence/absence of a grid does not agree with the result of selection stored in the RAM, for example, in the case in which the normal process or the simplified process is selected although a grid is used, the controller 101 may notify this to a user or may control to inhibit the radiation source 2 from emitting radiation. For example, the notification may be shown on the examination screen 131 (on a screen of the display 103), or may be output by a voice sound, vibrations, or the like. This can prevent execution of an unnecessary scattered radiation removal process.

The mobile radiographic apparatus 10 may include a device for detecting alignment error between the radiation source 2 and the FPD cassette 3. In this case, a threshold for detecting the alignment error may be automatically switched depending on whether the photographing is to be performed by using a grid. In one example in which photographing is performed by not using a grid, the threshold for detecting the alignment error may be increased to be higher than that for photographing using a grid (so as to loosen the detection criterion or extend the acceptable range). In the absence of a grid, there is no effect of moiré fringes, and therefore, the acceptable range is wide. In this embodiment, for dynamic image photography that is performed without a grid, this processing (scattered radiation removal control processing “A”) is conducted. However, this processing may also be performed in photography with a grid. In this case, in a condition in which performing the scattered radiation removal process is selected, that is, photographing is performed without a grid, the threshold for detecting the alignment error may be increased to be higher than that in a case of selecting not performing the scattered radiation removal process (so as to loosen the detection criterion or extend the acceptable range).

In response to operation to the exposure switch 102a, the controller 101 makes the drive unit 106 cause emission of radiation to a subject “H” from the radiation source 2, under the set irradiation conditions. The FPD cassette 3 accumulates and reads radiation that is emitted, in synchronization with the radiation source 2, and it generates image data of a radiographic image (still image or dynamic image) and transmits the image data to the main body 1.

After the first communication unit 105a receives (acquires) the dynamic image from the FPD cassette 3, the controller 101 executes the processes in step S2 and the subsequent steps.

The controller 101 may conduct the following process before advancing the processing to step S2.

A fast Fourier transform (FFT) analysis is performed on the received dynamic image. In a case in which a power spectrum value of a frequency corresponding to a grid moiré fringe exceeds a predetermined threshold, the image is determined as being obtained by photographing using a grid. The frequency corresponding to a grid moiré fringe and the threshold are stored in the storage 104 in advance. In addition, the FFT analysis is performed on a predetermined frame image of the dynamic image, for example, a first frame image, and the determination is performed on this frame image. The image may be determined as being obtained by photographing using a grid. In this case, in the condition that the result of selection of the scattered radiation component removal process stored in the RAM is the normal process or the simplified process, a confirmation screen is popped up on the examination screen 131 shown on the display 103. The confirmation screen shows a message such as “The image was obtained by photographing using a grid. Are you sure you want to execute the scattered radiation component removal process?”, an “EXECUTE” button, and a “CANCEL” button. The controller 101 keeps the result of selection of the scattered radiation component removal process stored in the RAM, as it is, in response to the “EXECUTE” button being pressed down. On the other hand, the controller 101 changes the result of selection of the scattered radiation component removal process stored in the RAM, to the process that does not involve the scattered radiation removal process, in response to the “CANCEL” button being pressed down. This can reduce waste of processing time.

In step S2, the controller 101 determines whether to perform the scattered radiation removal process, based on the result of selection stored in the RAM (step S2).

In the case in which the normal process or the simplified process is selected, it is determined to perform the scattered radiation removal process. In the case in which the process that does not involve the scattered radiation removal process is selected, it is determined to not perform the scattered radiation removal process.

Upon determining to not perform the scattered radiation removal process (step S2; NO), the controller 101 advances the processing to step S6.

Upon determining to perform the scattered radiation removal process (step S2; YES), the controller 101 selects a target frame image to be subjected to the scattered radiation removal process (step S3).

For example, the controller 101 selects all of frame images of a dynamic image that is obtained by photographing, as processing-target frame images.

In another example, only one or some of frame images of a dynamic image that is obtained by photographing may be selected as targets to be subjected to the scattered radiation removal process.

Specifically, frame images in which unstable radiation output and a very low image signal are expected in advance, such as a radiation emission start frame and a radiation emission finish frame, may be excluded, whereas the remaining frame images may be selected as processing-target frame images. In this case, the frame number of the frame image in which a very low image signal is expected in advance, is preliminarily set.

In another case, a frame image that is specified by a user using a user interface (UI) or the like, may be selected as a processing target. The frame images may be specified by designating a start frame and a finish frame of processing targets, as illustrated in FIG. 7. Moreover, for example, only the first frame image, the first or the last 100 frame images, 50% of all frame images, which are obtained in the middle between the start frame and the finish frame, or other frame images, may be specified. In addition, frame images may be discretely specified.

In yet another case, the main body 1 may receive a signal that is synchronous with movement of a patient, such as an exhalation/inhalation signal of a respirator or myogenic potential, at the time of photographing. In this case, a frame image that is obtained after this signal being synchronous with the movement of the patient is received, may be selected as a processing-target frame image.

In yet another case, an instruction for starting movement may be notified to a patient in photography, by sound represented by automatic voice, by displaying an image, by vibration such as of a portable vibration function, or by other means. In this case, notification start timing may be obtained by the main body 1, and a frame image corresponding to a frame image that is obtained after the notification is started, may be selected as a processing-target frame image.

In yet another case in which the order information from the RIS 30 contains a frame number of a processing-target frame image, the frame image of this frame number may be selected as a processing-target frame image.

Specifying only one or some of frame images of a dynamic image as the processing-target frame images, can reduce the processing time for the scattered radiation removal process.

Next, the controller 101 selects the method of the scattered radiation removal process (step S4).

Herein, the method of the scattered radiation removal process is selected based on the result of selecting the scattered radiation component removal process stored in the RAM. That is, one of the normal process and the simplified processes (simple body thickness estimation and simple scattered radiation component estimation) is selected.

Thereafter, the controller 101 executes the scattered radiation removal process on the selected processing-target frame image by using the selected method (step S5), and it then advances the processing to step S6.

In the case of executing the scattered radiation removal process in step S5, the controller 101 stores the dynamic image before the process is performed, in the storage 104.

The basic process of the scattered radiation removal process can use a publicly known method that is disclosed in, for example, JP 2019-126524A or JP 2019-129988A. For example, a body thickness of a subject is estimated based on irradiation conditions, such as tube voltage, an exposure dose, and a photographing distance, and a signal value of each pixel of a frame image. Then, scattered radiation components of each pixel of a radiographic image are estimated based on the estimated body thickness and are removed (subtracted) from the radiographic image.

In the case in which the selected method of the scattered radiation removal process is the normal process, the controller 101 executes the above-described basic processes on every processing-target frame image of the dynamic image. The basic processes include body thickness estimation based on the processing-target frame image, scattered radiation component estimation, and subtraction of scattered radiation components from the processing-target frame image. In this case, an estimated value of a frame image that is being processed may be separated from estimated values of the previous and the next frame images. This estimated value of the frame image that is being processed may be determined as being abnormal, and a correction process may be performed based on the estimated values of the previous and the next frame images.

In the case in which the selected method of the scattered radiation removal process is the simplified process (simple body thickness estimation), the controller 101 performs the body thickness estimation on one or some of the preliminarily set frame images, among the processing-target frame images, based on the one or some of the frame images. Moreover, the controller 101 calculates (obtains) a body thickness of each of the other frame images, from the result of the body thickness estimation of the frame image other than the frame image that is being processed. For example, the result of estimating the body thickness of other frame image is copied. In one case in which a differential signal value between adjacent frame images is close to ±0 due to little movement of a patient, the result of estimating the body thickness of the previous or the next frame image is copied.

Alternatively, an average, a median value, or the like of the results of estimating the body thickness of a plurality of frame images may be calculated, and the resultant value may be used as a body thickness of a frame image that is still not subjected to the body thickness estimation. In this case, the estimation result to be used in calculation of body thickness is desirably an estimation result that is calculated from the frame image temporally close to the frame image that is being processed. In another example, on the basis of the time relationship between a frame image to be calculated and a frame image being an estimation source, the result of estimating the body thickness may be weighted (for example, by using a Gaussian function), and an average of a plurality of the weighted estimation results may be calculated as the body thickness. In addition, the calculation method may be changed for each frame rate. Then, the scattered radiation component estimation is performed on each processing-target frame image, based on the calculated (estimated) body thickness, and the estimated scattered radiation components are subtracted from the corresponding frame image.

The body thickness estimation of each processing-target frame image may be simplified by one of the following methods.
A table in which gender, height+weight, BMI, or a combination of two or more thereof, and a body thickness is associated with each other, is preliminarily stored in the storage 104, and a body thickness is estimated based on the table and the patient information of a patient who is photographed by dynamic image photography.
A result of estimating a body thickness in a still image that is obtained by photographing in the same examination is used.
The photographing distance (SID) that is contained in the photographing conditions, and an SSD (distance from a tubular lamp to a patient surface) that is measured by a distance measurement sensor provided to the radiation source 2, are used to calculate an equation “SID - SSD = body thickness”.
Information of a patient body shape button (e.g., small, medium, or large for child, and small, medium, or large for adult) selected by user operation or the like in photography is used in the body thickness estimation.

In the case in which the selected method of the scattered radiation removal process is the simplified process (simple scattered radiation component estimation), the controller 101 performs the scattered radiation component estimation (including the body thickness estimation) on one or some of the preliminarily set frame images, among the processing-target frame images, based on the one or some of the frame images. Moreover, the controller 101 subtracts the estimated scattered radiation components from the one or some of the frame images. For the other frame images, a result of estimating the scattered radiation components from a frame image other than a frame image that is being processed, is subtracted from the frame image that is being processed. In the scattered radiation component estimation, an average, a weighted average, a median value, or the like of results of estimating the scattered radiation components in a plurality of frame images may be calculated. This calculation has a side effect that suppresses an increase in noise specific to the scattered radiation removal process. In view of this, in a situation in which an increase in processing time is acceptable, it is preferable to intentionally estimate scattered radiation components by using a plurality of frame images. In the case in which the estimation of scattered radiation components by using a plurality of frame images is scheduled, an exposure dose can be decreased at the time of photographing.

This embodiment is described by using an example in which the simple body thickness estimation or the simple scattered radiation component estimation can be selected as the simplified process. However, a process using the simple body thickness estimation and the simple scattered radiation component estimation, together, may also be selected. For example, the simple body thickness estimation is performed on an Nth frame image and on an N+1th frame image, and the simple scattered radiation component estimation is performed on frame images from an N+2th frame image to an N+5th frame image. In this manner, the simple body thickness estimation and the simple scattered radiation component estimation may be switched depending on the frame images.

In addition, the normal process may be performed on an ROI that is set, whereas one of the simplified processes may be performed on an image other than the ROI.

Moreover, a noise suppression process as disclosed in, for example, JP 2016-202219A or JP 2019-202019A, may also be performed on the dynamic image that has been subjected to the scattered radiation removal process. Alternatively or additionally, a density level difference reduction process as disclosed in JP 2019-129988A or the like may also be used together.

In quickly displaying an image, such as a preview image, the image may be displayed before the process is performed, even though the normal process or the simplified process is selected.

In step S6, the controller 101 performs an image process (dynamic analysis) on the dynamic image that has been subjected to the scattered radiation component removal process selected in step S1 (step S6).

A predetermined image process, such as a gradation process or a frequency process, is performed as the image process. For example, a user needs to check the analysis result of the dynamic analysis on the spot, such as in the case of the order information including information related to emergency medical care. In another example, the dynamic image is not transmitted to the dynamic analysis device 50. In such cases, the dynamic analysis that is specified by the order information is performed.

Then, the controller 101 shows the dynamic image that has been subjected to the image process, or analysis result, on the examination screen 131 on the display 103 (step S7), and it finishes the scattered radiation removal control processing “A”.

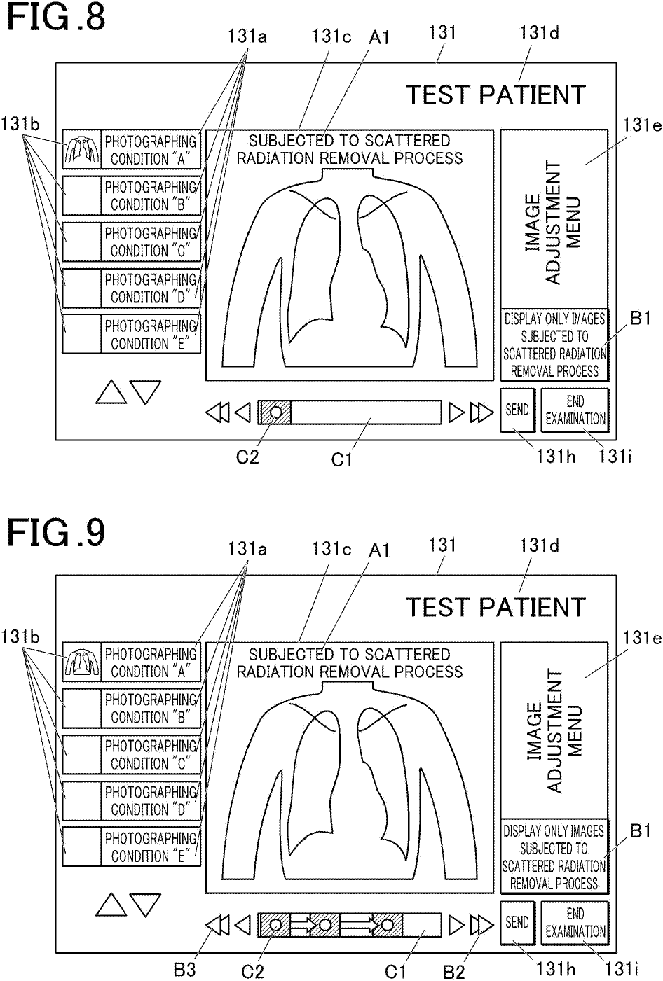

FIG. 8 illustrates an example of the examination screen 131 that is shown by the controller 101 in step S7. As illustrated in FIG. 8, the image display area 131c of the examination screen 131 shows a dynamic image that has been subjected to the image process or an analyzed image. Moreover, a thumbnail image of the image shown in the image display area 131c is displayed in the thumbnail display area 131b corresponding to the photographing condition key of the photography that has been performed. In addition, an image transmission button 131h is also displayed. This button is used for instructing transmission of the dynamic image that includes the displayed image, to an external device (PACS 40 or dynamic analysis device 50).

Information (characters or an icon) A1 showing whether the image is an image from which the scattered radiation components are removed (an image that has been subjected to the scattered radiation removal process), is superimposed on the image shown in the image display area 131c. This enables a user to check whether the displayed image has already been subjected to the scattered radiation removal process. In the state in which a dynamic image that has been subjected to the scattered radiation removal process (or analyzed image) is displayed, a switching button B1 is shown on the examination screen 131. It is possible to switch between displaying all of the frame images and displaying only the frame images that have been subjected to the scattered radiation removal process, by pressing down the switching button B1.

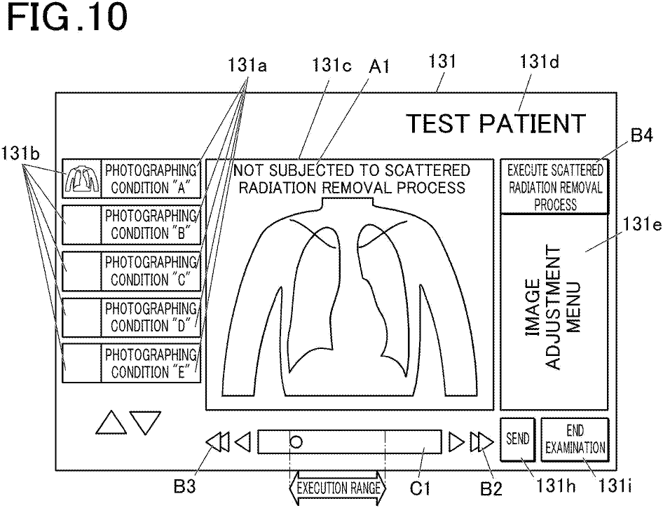

A seek bar C1 is provided on a lower side of the image display area 131c. The seek bar C1 shows a cursor C2 that shows a position of the currently displayed frame image in the entire dynamic image. The seek bar C1 shows a range of the frame images that have been subjected to the scattered radiation removal process and a range of the frame images that are not subjected to the scattered radiation removal process, which these ranges have different colors. In FIG. 8, the range of the processed frame images is shown by hatching, and the range of the unprocessed frame images is shown in white color (the same applies to FIGS. 9 and 10). This makes it possible for a user to easily understand the range of the frame images that have been subjected to the scattered radiation removal process and the range of the frame images that are not subjected to the scattered radiation removal process, in the dynamic image.

For the frame image that has been subjected to the scattered radiation removal process, a virtual grid condition (e.g., 6:1) that is performed, may be displayed as the information A1 that shows whether the scattered radiation removal process is already performed. The icon or the characters on the examination screen 131 may be changed in color or highlighted, depending on whether the scattered radiation removal process is already performed.

In one example, the frame images that have been subjected to the scattered radiation removal process may be discrete in the dynamic image, as illustrated in FIG. 9. In this case, pressing down a fast forward button B2 or a fast reverse button B3 may allow the display to be skipped to a closest frame image that has been subjected to the scattered radiation removal process.

For the dynamic image that is not subjected to the scattered radiation removal process, a UI for instructing execution of the scattered radiation removal process on the displayed dynamic image, may be provided. In one example, an execute button B4 for instructing execution of the scattered radiation removal process may be provided on the examination screen 131, as illustrated in FIG. 10. In this case, the scattered radiation removal process may be executed by pressing down the execute button B4. In response to pressing down the execute button B4, for example, a pop-up screen may be displayed in the same manner as in the change screen 132 illustrated in FIG. 7. This screen is used for setting the process (herein, the normal process, the simplified process (simple body thickness estimation), or the simplified process (simple scattered radiation component estimation)) to be used in the dynamic image, the range of processing-target frame images, etc. The scattered radiation removal process may be executed in accordance with the settings from this screen. Alternatively or additionally, the range of processing-target frame images may be specified on the seek bar C1.

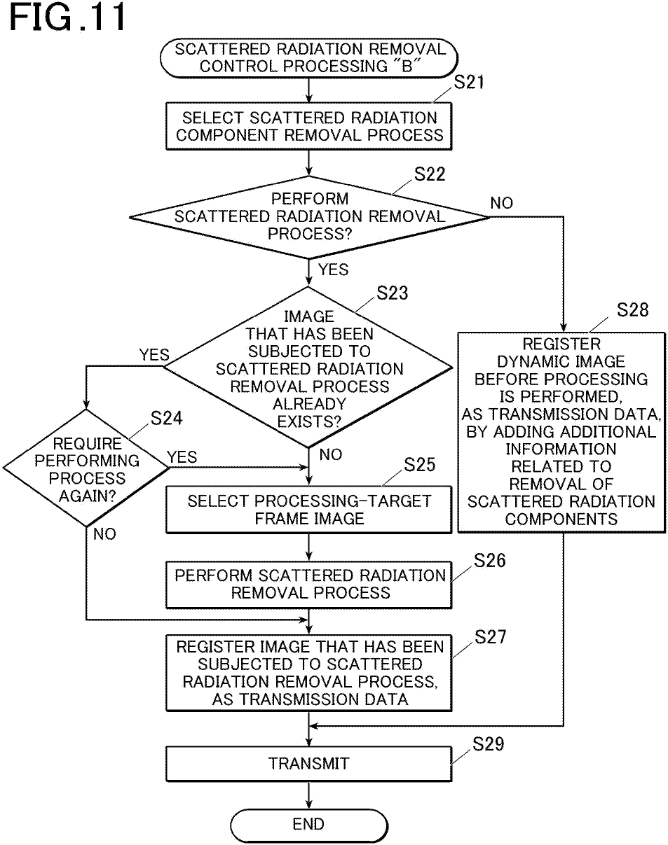

In response to pressing down the image transmission button 131h, the controller 101 executes the scattered radiation removal control processing “B” illustrated in FIG. 11, with respect to each image transmission destination. The controller 101 then transmits the dynamic image that has been subjected to the scattered radiation removal process or the dynamic image that is not subjected to the scattered radiation removal process, to the corresponding image transmission destination. The scattered radiation removal control processing “B” is executed by cooperation of the controller 101 and the program stored in the storage 104.

In the scattered radiation removal control processing “B”, first, the controller 101 selects the scattered radiation component removal process to be used in the dynamic image to be transmitted to the image transmission destination, from among the normal process, the simplified processes, and the process that does not involve the scattered radiation removal process (step S21).

Specifically, the controller 101 refers to the item of the image transmission destination (“IMAGE TRANSMISSION DESTINATION: PACS” or “IMAGE TRANSMISSION DESTINATION: IWS” in FIG. 4) in the order corresponding to the photographing condition button 131a that has been pressed down, in the employed process selection table 104b. In the case in which the information in this item is the term “ON_1”, the controller 101 selects the normal process.

In the case in which the information in this item is the term “ON_2_1”, the simplified process (simple body thickness estimation) is selected. In the case in which the information in this item is the term “ON_2_2”, the simplified process (simple scattered radiation component estimation) is selected. In the case in which the information in this item is the term “OFF”, the process that does not involve the scattered radiation removal process is selected. Thereafter, the result of selection is stored in the RAM.

For example, the image transmission destination may be an external device that does not perform the scattered radiation removal process and an analysis process based on an image signal value, such as the PACS 40. In this case, the term “ON_1”, the term “ON_2_1”, or the term “ON_2_2” is stored in the employed process selection table 104b, and one of the normal process and the simplified processes is selected.

On the other hand, for example, the image transmission destination may be an external device that can perform image analysis, such as the dynamic analysis device 50. Some image analysis is performed without the need for the scattered radiation removal process. The scattered radiation removal process is a process of increasing image noise, in principle, and therefore, some types of dynamic analysis may adversely affect the analysis due to effect of the image noise. From this point of view, it is desirable to transmit an image that is not subjected to the scattered radiation removal process and to allow processing the image in accordance with the purpose, at the image transmission destination. Thus, in one example in which the image transmission destination is an external device that can perform image analysis, such as the dynamic analysis device 50, the term “OFF” is stored in the employed process selection table 104b, and therefore, the process that does not involve the scattered radiation removal process is selected.

Then, the controller 101 determines whether to perform the scattered radiation removal process, based on the result of selection stored in the RAM (step S22).

The controller 101 determines to perform the scattered radiation removal process, in the state in which the normal process or the simplified process is selected in step S21.

Upon determining to perform the scattered radiation removal process (step S22; YES), the controller 101 determines whether an image that has been subjected to the scattered radiation removal process already exists (step S23).

In the case of determining that an image that has been subjected to the scattered radiation removal process does not exist (step S23; NO), the controller 101 advances the processing to step S25.

In the case of determining that an image that has been subjected to the scattered radiation removal process already exists (step S23; YES), the controller 101 determines whether to require performing the process again (step S24).

In one example in which there are only images that have been subjected to the simplified process, although the normal process is selected in step S21, it is determined that the process must be performed again.

Upon determining that the process must be performed again (step S24; YES), the controller 101 advances the processing to step S25.

In step S25, the controller 101 selects a processing-target frame image (step S25).

The process in step S25 is similar to that described in relation to step S3 in FIG. 6, and therefore, the above-described descriptions are referred to for this process.

Thereafter, the controller 101 executes the scattered radiation removal process selected in step S21, on the processing-target frame image selected in step S25 (step S26).

The process in step S26 is similar to that described in relation to step S5 in FIG. 6, and therefore, the above-described descriptions are referred to for this process.

Next, the controller 101 registers the dynamic image that has been subjected to the scattered radiation removal process, as transmission data (step S27). The controller 101 then makes the second communication unit 105b transmit the transmission data to the external device of the image transmission destination (step S29), and it finishes the scattered radiation removal control processing “B”.

On the other hand, upon determining that the process does not need to be performed again in step S24 (step S24), the controller 101 registers the dynamic image that has been subjected to the scattered radiation removal process, as transmission data (step S27). The controller 101 then makes the second communication unit 105b transmit the transmission data to the external device of the image transmission destination (step S29), and it finishes the scattered radiation removal control processing “B”.

On the other hand, upon determining to not perform the scattered radiation removal process in step S22 (step S22; NO), the controller 101 registers the dynamic image, as transmission data, by adding additional information necessary for the external device to remove scattered radiation components from the dynamic image (original image) that is not subjected to the scattered radiation removal process (step S28). The additional information relates to removal of scattered radiation components, and it is added to, for example, a digital image and communications in medicine (DICOM) tag. Then, the controller 101 makes the second communication unit 105b transmit the transmission data to the external device of the image transmission destination (step S29), and it finishes the scattered radiation removal control processing “B”.

The additional information that is added to the dynamic image in step S28 includes, for example, information showing whether the scattered radiation removal process is already performed, and irradiation conditions at the time of photographing (e.g., tube voltage, an exposure dose, a photographing distance, tube current, irradiation time, frame rate, and grid information). In addition, process parameters such as information of body thickness and scattered radiation components, may also be included to the additional information. The information of body thickness that is added as the additional information may be calculated from an image or may be calculated by other simple method. This additional information makes it possible for the image transmission destination to perform the scattered radiation removal process on the dynamic image.

The scattered radiation removal process is a process that requires a long time, as described above. Thus, continuously transferring the dynamic image can cause waiting for execution of the scattered radiation removal process, at the image transmission destination. On the other hand, an urgent dynamic image, such as for emergency medical care, should be preferentially processed. In view of this, at the time of transmitting the image, a flag that shows presence/absence of urgency may be added as the additional information for the dynamic image. Under these conditions, the external device of the image transmission destination, which receives a dynamic image to which the flag showing the presence of urgency is added, may skip other dynamic image that waits for execution of the scattered radiation removal process, and it may start performing the scattered radiation removal process on the urgent dynamic image. In addition, in the case in which the flag showing the presence of urgency is added, processes may be performed by prioritizing the scattered radiation removal process and other analysis items, such as dynamic analysis.

For a dynamic image that is obtained by photographing using a grid, instead of performing the scattered radiation removal process, the image process and a necessary dynamic analysis based on the order information are performed, and the dynamic image and the analysis result are then transmitted to the image transmission destination.

There has been no study of the necessity of performing the scattered radiation removal process on every frame image of a dynamic image before they are used in dynamic analysis, heretofore. In such a situation, the inventors have found that it is not necessary to perform the scattered radiation removal process on every frame image of a dynamic image, depending on the type of dynamic analysis, etc. Still images are generally used in the condition that they are subjected to the scattered radiation removal process or they are obtained by photographing by inserting a grid. On the other hand, a dynamic image may be subjected to a dynamic analysis involving calculation of a difference between frame images. In view of this, the inventors have also found that it is not necessary to perform the scattered radiation removal process on every frame image of a dynamic image that is obtained by photographing without a grid, unlike still images.

As described above, the controller 101 of the mobile radiographic apparatus 10 selects the scattered radiation component removal process to be used in a dynamic image that is obtained by dynamic image photography, based on the order information of the dynamic image photography. For example, the scattered radiation component removal process to be used in a dynamic image is selected from among the followings: the normal process for removing scattered radiation components from the dynamic image, the simplified processes for removing scattered radiation components from the dynamic image by a method simplified more than the first process, and the process that does not remove scattered radiation components from the dynamic image.

This enables selecting the scattered radiation component removal process appropriate for the dynamic image. Thus, it is possible to suppress a reduction in working efficiency of a radiographer and a diagnosis doctor and an increase in waiting time of a patient who is ready to be photographed, which situations may occur due to generation of unnecessary processing time of the scattered radiation removal process. In addition, it is also possible to prevent undesirable deterioration in image quality, which is caused by performing an unnecessary scattered radiation removal process.

Note that the contents described in relation to the foregoing embodiment are preferable examples of the present invention, and the present invention should not be limited thereto.

For example, the embodiment is described above by using an example in which the dynamic image processing device of the present invention is employed in the mobile radiographic apparatus. However, the present invention may be used in a console of a stationary radiographic apparatus or the like.

The embodiment is described above on the assumption that one of the normal process, the simplified processes, and the process that does not involve the scattered radiation removal process is selected based on the order information, as the scattered radiation component removal process to be used in a dynamic image. However, the selection choices are not limited thereto. In one example, one of the normal process and the process that does not involve the scattered radiation removal process, can be selected as the scattered radiation component removal process to be used in a dynamic image. Alternatively, one of the normal process and the simplified processes can be selected as the scattered radiation component removal process to be used in a dynamic image. In addition, the process that can be selected as the scattered radiation component removal process may include other process. That is, the choices of the scattered radiation component removal process to be used in a dynamic image include the normal process, and at least one of the simplified processes, which are simplified more than the normal process, and the process that does not involve the scattered radiation removal process.

In the above-described embodiment, the scattered radiation component removal process is selected based on the employed process selection table; however, it may be selected based on a photographing condition key, a frame rate, a pixel size, a total number of frames obtained by photographing, a photographing condition (kV, ms, or mA), or the like.

The above describes an example of using a hard disk drive, a semiconductor nonvolatile memory, or the like, as a computer-readable medium for the program of the present invention. However, the medium is not limited thereto.

Other computer-readable medium, for example, a portable recording medium such as a CD-ROM, can also be used. In addition, a carrier wave can be used as a medium that provides data of the program of the present invention via a communication line.

In addition, details of the structure and details of the operation of each device that constitutes the dynamic image processing system can also be modified or altered within a range not departing from the gist of the present invention, as appropriate.

Although embodiments of the present invention have been described and illustrated in detail, the disclosed embodiments are made for purposes of illustration and example only and not limitation. The scope of the present invention should be interpreted by terms of the appended claims.

What is claimed is:

1. A dynamic image processing device comprising:
a receiver configured to receive order information of dynamic image photography;
an acquirer configured to acquire a dynamic image that is obtained by performing the dynamic image photography; and
a hardware processor configured to select a scattered radiation component removal process to be used in the dynamic image, based on the order information.

2. The dynamic image processing device according to claim 1, wherein the scattered radiation component removal process includes a first process for removing scattered radiation components from the dynamic image, and at least one of a second process for removing scattered radiation components from the dynamic image by a method simplified more than the first process and a third process that does not remove scattered radiation components from the dynamic image.

3. The dynamic image processing device according to claim 2, wherein the order information includes at least one of information related to a type of dynamic analysis and information related to a type of diagnosis, and
the hardware processor is further configured to select the scattered radiation component removal process to be used in the dynamic image, based on at least one of the type of dynamic analysis and the type of diagnosis.

4. The dynamic image processing device according to claim 3, wherein the hardware processor is further configured to select the first process in a case in which the information related to the type of dynamic analysis includes information related to microscopic analysis.

5. The dynamic image processing device according to claim 3, wherein the hardware processor is further configured to select the first process in a case in which the information related to the type of diagnosis includes information related to emergency medical care.

6. The dynamic image processing device according to claim 3, wherein the scattered radiation component removal process includes the first process and the third process, and
the hardware processor is further configured to select the third process in a case in which the information related to the type of dynamic analysis includes information related to macroscopic analysis.

7. The dynamic image processing device according to claim 3, wherein the scattered radiation component removal process includes the first process and the third process, and
the hardware processor is further configured to select the third process in a case in which the information related to the type of diagnosis does not include information related to emergency medical care.

8. The dynamic image processing device according to claim 2, further comprising a transmitter, wherein
the scattered radiation component removal process includes the first process and the third process, and
the transmitter is configured to transmit information related to removal of scattered radiation components and to transmit the dynamic image, to an external device of a transmission destination of the dynamic image, in a case in which the hardware processor selects the third process.

9. The dynamic image processing device according to claim 2, wherein the hardware processor is further configured to remove scattered radiation components from the dynamic image, in a case in which the first process or the second process is selected, and
the hardware processor is further configured to remove the scattered radiation components by using one or some of frame images of the dynamic image.

10. The dynamic image processing device according to claim 9, wherein the hardware processor is further configured to remove scattered radiation components from only one or some of frame images of the dynamic image.

11. The dynamic image processing device according to claim 9, wherein the hardware processor is further configured to: in a case of selecting the second process,
acquire a parameter for removing scattered radiation components, based on one or some of frame images of the dynamic image; and
remove scattered radiation components from each target frame image, from which scattered radiation components are to be removed, of the dynamic image by using the acquired parameter.

12. The dynamic image processing device according to claim 1, further comprising a display configured to display the dynamic image, wherein
the display is further configured to display information indicating whether the displayed dynamic image is an image from which scattered radiation components are removed, with respect to each frame image of the dynamic image.

13. A dynamic image processing system comprising a first dynamic image processing device and a second dynamic image processing device, wherein
the first dynamic image processing device is the dynamic image processing device according to claim 1, and
the second dynamic image processing device is configured to remove scattered radiation components from a dynamic image that is transmitted from the first dynamic image processing device, based on the dynamic image and information related to removal of scattered radiation components.

14. The dynamic image processing system according to claim 13, wherein the first dynamic image processing device is mounted on a mobile radiographic apparatus.

15. A non-transitory recording medium storing a computer-readable dynamic image processing program, the dynamic image processing program related to removal of scattered radiation components from a dynamic image that is obtained by dynamic image photography, the dynamic image processing program configured to cause a computer to execute:
receiving that is receiving order information of the dynamic image photography;
acquiring that is acquiring the dynamic image that is obtained by performing the dynamic image photography; and
selecting that is selecting a scattered radiation component removal process to be used in the dynamic image, based on the order information.

16. The recording medium according to claim 15, wherein the scattered radiation component removal process includes a first process for removing scattered radiation components from the dynamic image, and at least one of a second process for removing scattered radiation components from the dynamic image by a method simplified more than the first process and a third process that does not remove scattered radiation components from the dynamic image.

17. The recording medium according to claim 16, wherein the order information includes at least one of information related to a type of dynamic analysis and information related to a type of diagnosis, and
the selecting is configured to select the scattered radiation component removal process to be used in the dynamic image, based on at least one of the type of dynamic analysis and the type of diagnosis.

18. A dynamic image processing method related to removal of scattered radiation components from a dynamic image that is obtained by dynamic image photography, the method comprising:
receiving that is receiving order information of the dynamic image photography;
acquiring that is acquiring the dynamic image that is obtained by performing the dynamic image photography; and
selecting that is selecting a scattered radiation component removal process to be used in the dynamic image, based on the order information.

19. The dynamic image processing method according to claim 18, wherein the scattered radiation component removal process includes a first process for removing scattered radiation components from the dynamic image, and at least one of a second process for removing scattered radiation components from the dynamic image by a method simplified more than the first process and a third process that does not remove scattered radiation components from the dynamic image.

20. The dynamic image processing method according to claim 19, wherein the order information includes at least one of information related to a type of dynamic analysis and information related to a type of diagnosis, and
the selecting is configured to select the scattered radiation component removal process to be used in the dynamic image, based on at least one of the type of dynamic analysis and the type of diagnosis.
